(19) United States
(12) Patent Application Publication
(10) Pub. No.: US 2021/0373015 A1
(43) Pub. Date: Dec. 2, 2021

(54) KITS AND METHODS FOR REAL-TIME MULITPLEX DETECTION AND IDENTIFICATION OF PATHOGENS

(71) Applicant: Myriad Applied Technologies, Inc., Washington, DC (US)

(72) Inventors: DAVE JOHNSON, WASHINGTON, DC (US); AARON URIAH MISHLER, WACCABUC, NY (US)

(73) Assignee: Myriad Applied Technologies, Inc., Washington, DC (US)

(21) Appl. No.: 17/288,308

(22) PCT Filed: Oct. 23, 2019
(86) PCT No.: PCT/US2019/057592

§ 371 Date: Apr. 23, 2021

Related U.S. Application Data
(60) Provisional application No. 62/749,516, filed on Oct. 23, 2018.

(51) Int. Cl. G01N 33/569; G01N 33/543

(52) U.S. Cl. CPC G01N 33/56911; G01N 33/54333

(57) ABSTRACT

The invention encompasses kits and rapid methods for real-time multiplex detection and identification of one or more pathogens using glass well plates that include chemically modified surfaces to capture the presence of one or more pathogens from a small sample and magnetic microbeads with modified surfaces for capturing of pathogens within a volume of sample and collecting with an external magnet.

### FIELD OF THE INVENTION

The invention encompasses kits and rapid methods for real-time multiplex detection and identification of one or more pathogens using glass well plates that include chemically modified surfaces to capture the presence of one or more pathogens from a small sample and magnetic microbeads with modified surfaces for capturing of pathogens within a volume of sample and collecting with an external magnet.

### BACKGROUND OF THE INVENTION

Clinicians and field health care workers have long desired portable diagnostic tools that rapidly provide multiple differential diagnostic detection and identification data in order to implement targeted therapeutic intervention and monitor trends in disease transmission through epidemiological surveillance. Several diagnostic tests including nucleic acid-based multiplex isothermal amplification methods have been developed and used to detect various pathogens. These detection formats, including some isothermal platforms, are rigid and inflexible and do not allow for rapid simultaneous detection and identification of pathogens. Such platforms hardly enable simultaneous identification and quantitation of a pathogen load on a surface or in an infected person.

The present invention encompasses kits and methods that overcome the shortcomings of current diagnostic methods and protocols. The present invention encompasses kits and methods that provide a versatile and flexible pathogen detection platform that enables real-time multiplexing for rapid detection, identification, and quantitation of bacteria, viruses, and protozoans, and other pathogens. The invention also encompasses commercially practicable diagnostic kits and methods of detection that are portable, inexpensive, and field-deployable. Furthermore, the invention encompasses a sensitive and specific multiplexing detection system that will contribute to national and global public health safety, contribute to ensuring early diagnosis of multiple infectious diseases and simultaneous identification of their causative agents, and enable therapeutic intervention.

### SUMMARY OF THE INVENTION

The invention encompasses kits and rapid methods for real-time multiplex detection and identification of one or more pathogens using glass well plates that include chemically modified surfaces to capture the presence of one or more pathogens from a small sample and magnetic microbeads with modified surfaces for capturing of pathogens within a volume of sample and collecting with an external magnet.

In one embodiment, the invention encompasses diagnostic tool for simultaneous detection of multiple pathogens. In certain embodiments, the diagnostic tool is a portable.

In certain embodiments, the invention encompasses kits and methods for the detection, identification, and/or quantification of pathogens including glass well plates that can incorporate chemically modified surfaces to capture the presence of bacteria from a small sample.

In certain embodiments, the invention encompasses kits and methods for the detection, identification, and/or quantification of pathogens including magnetic microbeads with modified surfaces for capturing of bacteria while agitated (e.g., mixed/shaken) with a small volume of sample, and then collected with a small external magnet. In certain embodiments, software will correlate the detected pathogen against a database of known pathogens for rapid identification.

In certain embodiments, the invention encompasses diagnostic kits and methods including glass plates with chemically modified surfaces and magnetic microbeads with modified surfaces. In certain embodiments, both are used to detect and identify the presence of a bacterial pathogen. In other embodiments, the detection and identification is used in various environments as set forth herein.

In certain embodiments, the invention encompasses diagnostic kits and methods encompassing: (1) glass well plate with chemically modified surfaces to capture bacteria to the well bottoms and walls, and (2) magnetic microbeads with modified surfaces for capturing of bacteria while mixed/shaken with a small volume of sample, and then collected with a small external magnet.

In certain embodiments, the invention includes wells with the tested sample that are filled and emptied (one or several times) then imaging each well while adding the small volume of the photoprobe solution. In certain embodiments, the same photoprobe to multiple wells to increase sensitivity (e.g. improve the detection), or different probes to different wells to increase the certainty of identification of specific pathogens (e.g., improve identification).

In certain embodiments, the well plates are widely used for many methodologies and technologies in bioanalytical and clinical labs.

In other embodiments, a magnetic-bead approach prove more powerful for capturing small amount of bacteria in relatively large sample volumes. In certain embodiments, the volume of the sample (e.g., 0.01 mL, 1 mL, 10 mL or even larger), is not necessarily a limit. Statistically, to detect a small amount of pathogen cells distributed in a large volume, requires large volumes of sample to be tested, regardless the technique. That is, with 10 cells per litter, he chances that at least one of these cells will be in 10 mL (i.e., in 0.01 litter) of the sample. In certain embodiments, using superparamagnetic beads addresses this issue. For example, enough beads can be dispersed in the large volume of sample, and then placing a magnet on the outside of the sample container, the beads can be collected on the inside of the wall and the sample poured out of the container. Then the beads can be transferred in their concentrated form for imaging in a well plate. Indeed, the beads have to be superparamagnetic, that is, the can respond strongly even to weak magnetic fields, but cannot permanently magnetize (i.e., like ferromagnetic material) and thus do not clump together after the magnet is removed.

In certain embodiments, the dynamic detection provides a means for fast analysis of mixtures of bacteria and other pathogens.

In certain embodiments, the invention encompasses kits and methods for: (1) efficient capturing with high specificity of bacterial cells from a complex sample mixture, and (2) adding a solution a probe to the captured volume of potential pathogens while monitoring it continuously for a minute or even less.

Another embodiment encompasses a real-time multiplex, rapid, sensitive, and specific assay for simultaneous detection, identification, and quantification of pathogens

In certain embodiments, the pathogen includes, but is not limited to, human immunodeficiency virus (HIV), Ebola virus species (Zaire, Sudan, Tai-Forest, Reston, Bundibugyo), Marburg virus (MBV), Yellow fever virus (YFV), hepatitis-B virus (HBV), Lassa fever virus (LFV), Plasmodium species, hepatitis-C virus (HCV), hepatitis-E virus (HEV), dengue virus (DENV), Chikungunya virus (CHIKV), Japanese Encephalitis virus (JEV), Middle Eastern Respiratory Syndrome Corona virus (MERS CoV), Mycobacterium species (MTB), Severe Acute Respiratory Syndrome Corona virus (SARS CoV), West Nile (WNV) virus, Cytomegalovirus (CMV), Parvovirus (PAB19), Plasmodium species (PLM), Leishmania species (LE), Trypanosoma species (TRY), Zika virus (ZKV) and an array of other infectious pathogens.

In certain embodiments, the diagnostic kits and methods are developed for laboratory as well as point-of-care and field application so as to enable differential pathogen diagnosis, blood donor screening, early diagnosis of infections, and monitoring of therapeutic efficacy.

In other embodiments, the kits and methods of the invention further encompasses a versatile and flexible platform that allows the detection components as duplex, triplex, and/or multiplex assays for real-time quantitative detection, quantification, and identification of multiple pathogens, and groups of phylogenetically related infectious organisms.

Another embodiment of the invention encompasses kits and methods capable of distinguishing different species, genotypes or serotypes of a pathogen.

In certain embodiments, detection of pathogens is achieved using kits and methods of the invention that simultaneously detect multiple, disparate pathogens with a single system for use in several different environments. In certain embodiments, the system will be portable and ruggedized and will be operational in various locations where sophisticated lab equipment may or may not be available. In other embodiments, the kits and methods can be used as a protocol to support personnel who need to assess and act on the presence of a pathogen (e.g., hospitals, food and water production and distribution facilities, restaurants, cruise ships, port of entry facilities and military facilities.)

In certain embodiments, the invention encompasses kits and methods for the efficient capturing with high specificity of bacterial cells from a complex sample mixture. In certain embodiments, the invention encompasses adding a solution to the captured volume of potential pathogens while monitoring it continuously for a short duration (e.g., a minute or less).

### BRIEF DESCRIPTION OF THE DRAWINGS AND FIGURES

FIG. 1 illustrates an exemplary, non-limiting, 12-well glass prototype of a Bacterial Identification Chip (BIC) for bacterial detection and identification with a footprint of a “regular” microscope slide (dimensions: 3″×1″×0.12″). The number and the size of the wells can be widely varied.

FIG. 2 illustrates an exemplary, non-limiting, (a) Prototype of a Bacterial Identification Chip (BIC). (b) Sampling half a liter of “contaminated” water. (c) Conducting dynamic stating at each well of BIC.

FIG. 3 illustrates an exemplary, non-limiting, microscope images of whole bovine blood spiked with vegetative bacteria and stained with ThT (30 μM) on mannose-PEG-coated glass. (a,b,c) Bright-field; & (d,e,f) fluorescence. (a,d) B. sphaericus; (b,e) P. aerugniosa; and (c,f) E. coli. (Scale bar=20 μm).

FIG. 4 illustrates an exemplary, non-limiting, fluorescence staining kinetics of vegetative bacterial cells with THIA (6 ∂M, λex=420 nm, λem=475 nm.

FIG. 5 illustrates exemplary, non-limiting, sequential images of B. subtilis cells captured on mannose-functionalized surface, and stained with 1 μM ThT. (a) Brightfield image; (b) no dye present; (c-i) images at 0, 0.1 0.2, 0.3, 0.4, 0.5, 0.6, 0.7 s, respectively, after the addition of ThT. (Scale bar=10 μm).

FIG. 6 an exemplary, non-limiting, technique for surface derivatization of a silica-based substrate, such as glass or SiO₂-coated silicon.

FIG. 7. Illustrates a ribbon representation of the structures of (a) GFP and (b) BCA with the lysine residues shown as ball-and-stick models.

FIG. 8 illustrates fluorescence spectra of GFP solutions (PBS buffer, pH=6.85) with different concentrations: (a) 20-500 nM and (b) 250 pM-5 nM (Aex=480 nm; Aem=495-650 nm). Dependence of the emission intensity at the maximum of the fluorescence band, F(Amax), on the protein concentration, CGFP, plotted against: (c) linear abscissa and (d) logarithmic abscissa. A linear function, F(Amax)=a+b CGFP, and Eq. (3) were used for fitting the data. Apparently, Eq. (3) yields a superior fit for this set of data that spans more than three orders of magnitude not only along the abscissa, but also along the ordinate.

FIG. 9 illustrates enzymatic activity of BCA, catalyzing the hydrolysis of 0.1 mM NPA in aqueous HEPES buffer (pH=8.25). (a) Homogeneous NPA hydrolysis, catalyzed by various concentrations of BCA dissolved in the solution. (b) Heterogeneous NPA hydrolysis, catalyzed by BCA immobilized on glass surfaces that were PEGylated at various RDM ratios. The glass slides were immersed in the NPA solution. (c) Dependence of the initial rate of hydrolysis, Vi, on RDM. The formation of NP was monitored via its absorption at 348 nm. The blank solution (i.e., CBCA=0) and the blank PEGylated slides (i.e., RDM=0) manifested the same rates of non-catalyzed NPA hydrolysis indicating that the increase in the rates, observed for RDM>0, is due solely to the surface-bound enzyme and not to other components on the surfaces of the PEGylated glass.

FIG. 10 illustrates (a) a suspension of superparamagnetically doped microbeads, dropped on coated glass surfaces in wells of polydimethylsiloxane (PDMS), (b) gravity-driven settling of the beads, (c) beads settled on the glass surface, and (d) beads pulled off the surface.

FIG. 11 illustrates removing of superparamagnetically doped polymer microbeads (3-μm diameter) from glass surfaces using 1.2-pN net force. From a suspension in an aqueous solution (100 mM phosphate buffer, pH 7), the beads were allowed to settle for ˜3 min. (a-d) Reflection microscopy images of beads coated with PEG-3000 (a and b) on glass slides coated with PEG-3000 and (c and d) on noncoated glass slides; (a and c) after settling on the surface and before applying force, and (b and d) after applying magnetic force for 5 s. (e) Time course of desorption of beads from glass surfaces induced by 1.2-pN net force. (CB-CS designates coated beads settled on coated surfaces; and NB-CS designates noncoated beads settled on coated surfaces.) The beads were tracked while in focus. The depth of field of the used objective was 6 μm, exceeding the bead size. Therefore, the beads were still in focus and tracked for a few seconds after desorption from the glass surface.

FIG. 12 illustrates Images of superparamagnetically doped polymer beads (3-μm diameter). (a and b) Scanning-electron micrographs and (c and d) epifluorescence micrographs of beads that were (a and c) not coated and (b and d) coated with PEG-3000. For the electron microscopy images, the beads were coated with platinum. The autofluorescence of the beads was used for the fluorescence imaging. The scale bars correspond to 2 μm.

### DETAILED DESCRIPTION OF THE INVENTION

The invention generally encompasses kits and methods for detecting bacterial, viral, and protozoan nucleic acids in a sample such as those on a surface or from a biological sample of a subject, for example, a surface or subject clinically suspected of being infected with said pathogen(s). The kits and methods include real-time, quantitative assays multiplexed for detection and identification of pathogens (and their genotypes, subtypes or serotypes) including, but not limited to, HIV, EBOV, MARV, HBV, HCV, CHIKV, MERS CoV, PAB19, CMV, JEV, MTB, HEV, DENV, SARS CoV, YFV, LFV, LE, TRY, ZKV and/or PLM. In some examples, the methods include real-time multiplex detection, quantitation, and differentiation of the genotypes in a sample and/or discriminating subtypes (e.g., HCV-1a, 1b, or 1c), HCV2 or HCV2 subtypes (such as HCV-2a, 2b, 2a/c, or 2c), HCV3 or HCV 3 subtypes (such as HCV-3a or 3b), HCV4 or HCV4 subtypes (such as HCV-4a, 4b, 4c, or 4d), HCV5 or HCV5 subtypes (such as HCV-5a), HCV-6 or HCV6 subtypes (such as HCV-6a or 6b) and HCV7.

In another embodiment, the kits and method include real-time multiplex isothermal detection, differentiation, and quantitation of genotypes (for example detecting and/or discriminating HBV-A, HBV-B, HBV-C, HBV-D, HBV-E, or HBV-F). In other embodiments, the kits and methods include real-time isothermal multiplex detection, differentiation, and quantitation of a sample and/or discriminating genotypes, (such as, for example, ZEBOV, SUDV, RESTV, TAFV, and BDBV). In further embodiments, the kits and methods include real-time isothermal multiplex detection, differentiation, and quantitation of any pathogens including, but not limited to, acillus anthracis (anthrax); ° Clostridium botulinum toxin (botulism); ∘Yersinia pestis (plague); Variola major (smallpox) and other related pox viruses; Francisella tularensis (tularemia); Viral hemorrhagic fevers; Arenaviruses; Junin, Machupo, Guanarito, Chapare; Lassa, Lujo; Bunyaviruses; Hantaviruses causing Hanta Pulmonary syndrome, Rift Valley Fever, Crimean Congo Hemorrhagic Fever; Flaviviruses—Dengue; Filoviruses—Ebola; Marburg; Burkholderia pseudomallei (melioidosis); Coxiella burnetii (Q fever); Brucella species (brucellosis); Burkholderia mallei (glanders); Chlamydia psittaci (Psittacosis); Ricin toxin (Ricinus communis); Epsilon toxin (Clostridium perfringens); Staphylococcus enterotoxin B (SEB); Typhus fever (Rickettsia prowazekii); Food- and waterborne pathogens; Bacteria; Diarrheagenic E. coli; Pathogenic Vibrios; Shigella species; Salmonella; Listeria monocytogenes; Campylobacter jejuni; Yersinia enterocolitica; Viruses; Caliciviruses; Hepatitis A; Protozoa; Cryptosporidium parvum; Cyclospora cayatanensis; Giardia lamblia; Entamoeba histolytica; Toxoplasma gondii; Naegleria fowleri; Balamuthia mandrillaris; Fungi; Microsporidia; Mosquito-borne viruses □West Nile virus (WNV); LaCrosse encephalitis (LACV); California encephalitis; Venezuelan equine encephalitis (VEE); Eastern equine; encephalitis (EEE); Western equine encephalitis (WEE); Japanese encephalitis virus (JE); St. Louis encephalitis virus (SLEV); Yellow fever virus (YFV); Chikungunya virus; Zika virus; Nipah and Hendra viruses; hantaviruses; Tickborne hemorrhagic fever viruses; Bunyaviruses⋅Severe Fever with Thrombocytopenia Syndrome virus (SFTSV), Heartland virus; Flaviviruses; Omsk Hemorrhagic Fever virus, Alkhurma virus, Kyasanur Forest virus; Tickborne encephalitis complex flaviviruses; Tickborne encephalitis viruses; Powassan/Deer Tick virus; Tuberculosis, including drug-resistant TB; Influenza virus; Other Rickettsias; Rabies virus; Prions; Coccidioides spp.; Severe acute respiratory syndrome associated coronavirus (SARS-CoV), MERS-CoV, and other highly pathogenic human coronaviruses; Bacterial vaginosis, Chlamydia trachomatis, cytomegalovirus, Granuloma inguinale, Hemophilus ducreyi, hepatitis B virus, hepatitis C virus, herpes simplex virus, human immunodeficiency virus, human papillomavirus, Treponema pallidum, Trichomonas vaginalis; Acanthamebiasis; Anaplasmosis; Australian bat lyssavirus; Babesia, atypical; Bartonella henselae; BK virus; Bordetella pertussis; Borrelia mayonii; Borrelia; miyamotoi; Ehrlichiosis; Enterovirus 68∘Enterovirus 71; Hepatitis C; Hepatitis E; Human herpesvirus 6; Human herpesvirus 8; JC virus; Leptospirosis; Mucormycosis; Poliovirus; Rubeola (measles); and Streptococcus, among others.

In certain embodiments, the kits and methods of the invention encompass covalent attachment of one or more proteins to glass and silica surfaces, chemically modified with polyethylene glycol (PEG). In certain embodiments, due to the sensitivity of glass surfaces to strong acids and bases, cation-catalyzed deprotection of surface-bound aldehydes is utilized instead of using strong acids. In certain embodiments, introduction of bifunctional polymers to the coatings allowed for covalent attachment of proteins to the PEGylated surfaces. In certain embodiments, spectroscopic studies indicated that the surface-bound proteins preserve their functionality. In certain embodiments, the surface concentrations of the proteins, however, were not linearly proportional to the molar fractions of the bifunctional PEGs.

In certain embodiments, bioinert interfaces that resist protein adsorption and cell adhesion are one example of a component in the development of biomaterials. Such nonfouling surfaces, however, are limiting for numerous biomedical applications, for which selected interactions with the bio-logical media are required. Alternatively, controlled derivatization of bioinert surfaces with small molecules, polypeptides, or oligo and poly-saccharides yields interfaces that mediate bio specific interactions and suppress nonspecific interactions.

In certain embodiments, physisorption of proteins onto solid substrates is a facile and expedient method for preparation of non-fouling and even, bioactive interfaces. Such nonspecific adsorption of proteins, however, can lead to their partial or complete denaturation resulting in losses in their functionality. Furthermore, physisorbed coatings are susceptible to loss or replacement of their components due to desorption or competitive binding. Therefore, covalent attachment of the surface coatings to the supporting substrates is a preferred approach for surface engineering.

In certain embodiments, chemical derivatization of surfaces with non-charged water-soluble oligomers and polymers, which do not contain hydrogen-bond donating groups, tends to produce bioinert interfaces. Observed exceptions to this general rule, such as formation of bioinert layers from zwitterionic and hydroxyl-terminated oligomers, however, indicate that this field of sur-face engineering is largely unexplored.

In certain embodiments, it is believed that the hydration of the PEG chains dictates its nonfouling characteristics. In aqueous media, PEG assumes helical conformation, in which the distance between neigh-boring ether oxygens, nm, is similar to the average separation between the oxygens in liquid water. This match in oxygen-oxygen distances favors the intercalation of the PEG chains into the hydrogen-bonding network of bulk water. The hydration of the PEG molecules, hence, “insulates” their hydrophobic ethylene groups without disrupting the bulk water structure. In aqueous media, therefore, biological molecules in a close proximity with the PEG chains do not truly experience the presence of the polymer. Covalent attachment of layers of poly- and oligo-ethylene glycols to various solid substrates, indeed, presents a broadly chosen approach for engineering of nonfouling interfaces. PEGylated surfaces demonstrate some of the highest protein resistance; they are relatively durable; and chemically, PEGs are relatively easy to manipulate. In certain embodiments, utilizing self-assembled monolayers (SAMs) of alkylthiols on gold surfaces for engineering of bioactive interfaces has gained significant popularity due to the simplicity of the used chemistry and to the availability of high-resolution structural information for such SAMs. Furthermore, engineering of bioactive interfaces over thin metal films has been a driving force for the development of biosensing applications based on surface plasmon resonance techniques.

In certain embodiments, surface engineering based on thiol chemistry on coinage metals can be utilized. The requirement for coating the substrates with gold, silver or another noble metal compromises the cost efficiency of thiol-chemistry procedures. The metal coatings add undesired opacity to transparent substrates. The susceptibility of sulfur-gold conjugates to oxidation tends to compromise the durability and structural integrity of alkylthiol SAMs. Therefore, if chemically possible, a direct attachment of the surface coatings to the supporting substrate (instead of using thin layers of gold or silver) present a preferred approach for engineering of bioactive interfaces for a broad number of applications.

In certain embodiments, the attachment of the enzymes to the PEGylated surfaces involves non-covalent inter-actions, such as proteins-ligand association or metal ion chelation. Such non-covalent bonds are the weakest links in the chains holding the biological macromolecules to the substrate surfaces. Furthermore, the size of the complexes, which can provide non-covalent interactions with acceptable strength (e.g., streptavidin-biotin), is quite large and can even exceed the size of the proteins that they hold to the surface (e.g., the molecular weights of avidin and streptavidin are about 60 and 67-68 kDa, respectively). Covalent bonds are significantly smaller and stronger than the non-covalent complexes used for biocompatible interfaces. Therefore, a goal is to covalently attach globular proteins to silica-based surfaces and to demonstrate that they preserve their functionality via enzymatic assays.

In certain embodiments, biotin-(strept)avidin interaction for non-covalent attachments, for example, provides bonding strength of about 0.8 eV (i.e., dissociation constant ranging between 1 and 100 fM). The energy of a single (sigma) covalent bond between carbon and carbon, carbon-nitrogen, and carbon-oxygen, on the other hand, ranges between about 140 and 150 kJ/mol, which corresponds to about 1.5 eV. This twofold difference between the energies of covalent and non-covalent bonding interactions, results in more than ten-orders-of-magnitude difference between their dissociation rate constants. Under external pulling forces typical for biological macromolecular and cellular systems, therefore, while the non-covalent complexes have finite lifetimes, the covalent bonds are practically indissociable.

In certain embodiments, biophotinic and bio-electronic engineering poses requirements for the development of bioactive coatings on materials such as silicate glasses and silicon. In certain embodiments, the invention encompasses a kit including a method for generation of protein-functionalized coatings directly anchored to the surfaces of glass and silicon (Scheme 1). In certain embodiments, components of the coatings are covalently attached to each other and to the substrate. In particular, the proteins are attached directly to the substrate via chains with predetermined lengths. The surface-bound proteins manifested activity similar to the activity of the same proteins when free in solution. Fluorescence measurements and enzymatic assays allowed us to determine the dependence of the protein surface concentrations on the composition of the polymer mixture used for the bioinert layers.

In certain embodiments, in order to assure densely packed coatings, chemical reactions that do not involve bulky intermediates in the procedures for the preparation of the bioinert PEG layers are used. In addition, a unique and unprecedented method for deprotection of aldehydes, bound to glass surfaces, under relatively mild non-acidic condition. Because glass surfaces are susceptible to strongly acidic media, the traditionally used mineral acids for aldehyde deprotection cannot be applied for treatment of organic coatings on glass substrates. The described surface derivatization encompasses methodology with the potential for a broad use for rational design and engineering of bioactive interfaces on silica-based materials.

The invention further encompasses kits and methods that may be used for any purpose for which real-time isothermal multiplex detection, differentiation, and quantitation of bacterial, viral, and protozoa is needed in a field, laboratory, or clinical setting for diagnostic and prognostic applications. The nucleic acids are isolated from appropriate clinical biological samples including, but not limited to, cells, tissues, blood, serum, plasma, urine, cerebrospinal fluid, nasopharyngeal aspirates, middle ear fluids, bronchoalveolar lavage, tracheal aspirates, sputum, vomitus, buccal swabs, vaginal swabs, stool, and rectal swabs. The samples can be directly used in amplification reaction. In further example, the samples are first treated with lysis buffer or heat-treated before application in reaction medium. In some examples, nucleic acids are isolated or extracted from the samples with various nucleic acid extraction methods known to one of skill in the art.

In certain embodiments, the disclosed kits and methods are highly sensitive and specific for real-time isothermal multiplex detection, differentiation, and quantitation of pathogens described herein. In some examples, the disclosed methods can detect presence of at least about 1 International Unit (IU equivalent to about 5 copies) of a pathogen described herein in a sample or reaction volume. In other examples, the disclosed methods can detect presence of at least about 1 copy of a pathogen (e.g. at least about 10 to 10⁶ or more copies) in a sample or reaction volume. In some examples, the disclosed methods can predict with a sensitivity of at least 80% and a specificity of at least 80% for presence of one or more of a pathogen in a sample, such as a sensitivity of at least 85%, 90%, 95%, or even 100% and a specificity of at least of at least 85%, 90%, 95%, or even 100%.

In another embodiment, the invention encompasses covalently grafted coatings of polyethylene glycol (PEG), with a molecular weight (MW) exceeding 1 kDa, that suppressed the adhesion of polymer microspheres to glass surfaces. In certain embodiments, the invention encompasses kits and methods encompassing an adhesion propensity of polymer microspheres to flat glass surfaces when coated with PEGs with different length, varying from about 22 to 450 repeating units and corresponding to MW from about 1000 to 20 000 Da. (The PEGs with different MW are designated in the text as PEG-“MW in Da”, i.e., PEG-1000 to PEG-20000). In certain embodiments, the invention encompasses kits and methods encompassing coatings of PEGs with MW ranging between 3 kDa and 10 kDa, that provided optimal suppression of the nonspecific adhesion. In certain embodiments, when the microbeads and the glass were coated with PEG-5000, less than 3% of the beads remained on the surface after applying 1.2 pN pulling force. In certain embodiments, when only the beads or the glass were coated (with PEG-3000), the nonspecific interactions were still prevalent. In certain embodiments, the invention encompasses kits and methods encompassing adding a noncharged surfactant, (e.g., TWEEN 20), only marginally improved the suppression of nonspecific interactions. In certain embodiments, the invention encompasses kits and methods encompassing hydrated PEGs for suppressing nonspecific interactions between micrometer-size objects at nanometer separation.

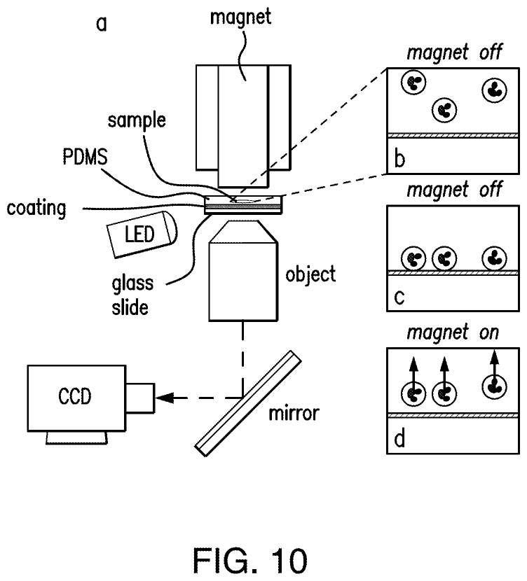

In certain embodiments, the invention encompasses kits and methods encompassing magnetic pullers, in order to characterize the nonspecific interaction between glass surfaces and superparamagnetically doped polymer microspheres (See FIG. 10).

In certain embodiments, the magnetic pullers usually employ a single electromagnet that does not generate a magnetic trap. In certain embodiments, the pullers are relatively simple devices, and they allow for a well-controlled exertion of relatively weak forces on magnetic micro- and nano-objects, (e.g., forces that are less than 10 pN), directed toward the magnet.

In certain embodiments, the magnetic pullers are inverted optical microscopes with electromagnets or permanent magnets above the sample focal plane. In certain embodiments, the microbeads that contain paramagnetic material are allowed to settle under gravity on the surface of a sample slide. In certain embodiments, the surface on which the beads settle is within the depth of field of the objective. In certain embodiments, the magnetic field gradients, generated by the magnet above the focal plane exert pulling forces on the beads. In certain embodiments, as the pulling force moves the microbeads away from the surface, and hence out of the depth of field, the beads “disappear” from the focus of the image. By recording movies of the beads settled in the field of depth, it is possible to monitor the number of beads on the glass surface at each time point.

In certain embodiments, superparamagnetic materials are used to ensure that (1) the beads attain complete magnetization in relatively weak fields and (2) their magnetization would not manifest hysteretic behavior when the field was turned off. Hence, the magnitude of the force depended mostly on the field gradient, which was readily controlled by varying the current passed through the electromagnet coil or by moving the permanent magnet up and down above the focal plane. Electromagnets were employed in order to avoid moving parts and eliminate unnecessary vibrations during the measurements.

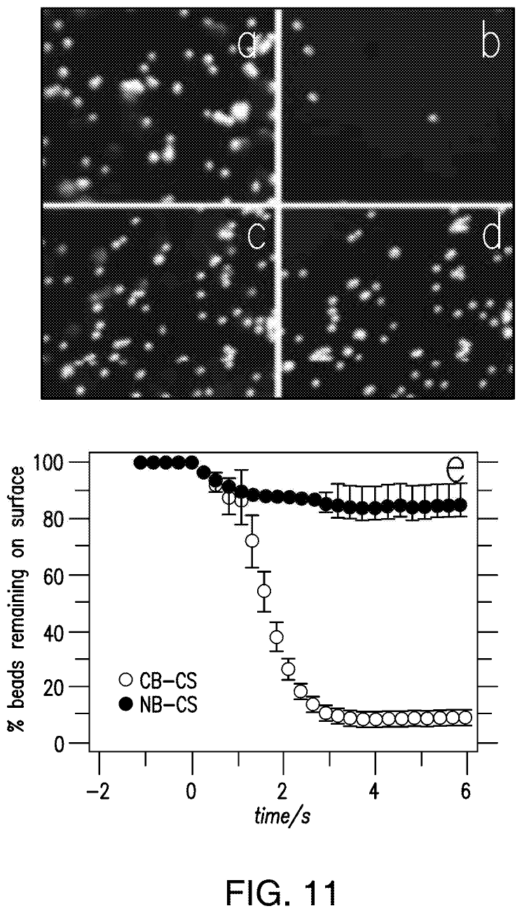

In certain embodiments, when suspended in the aqueous solution, each bead experienced ˜0.3 pN gravitational pull downward, as we determined from direct measurements and from calculations accounting for the bead buoyancy in the used media. After allowing the beads to settle on the glass surfaces for three minutes, the electromagnet is switched on to apply 1.2±0.3 pN upward net pulling force. We quantified the extent of the non-desired adhesion as the percent of beads that remained on the surface after applying the magnetic force for 5 s (FIG. 11).

In certain embodiments, in order to calibrate the magnetic puller, the objective was moved to attain a side view for the setup. A suspension of the superparamagnetic microbeads was introduced in a square capillary under the magnet. The velocities with which the microbeads in the capillary moved toward the magnet were recorded and employed the Stoke's drag equation for estimating the magnetic force on the beads at different distances from the magnet, and at various voltages applied to the coil of the electromagnet.

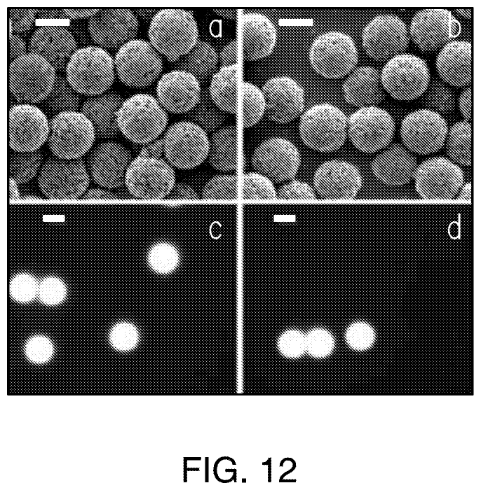

In certain embodiments, using surface-chemistry protocols glass slides were coated with PEGs with MW=1, 2, 3, 5, 10, and 20 kDa. Concurrently, resorting to carboxylated polymer microspheres (that were superparamagnetically doped) allowed PEGylation via aqueous-phase coupling protocols. FITR spectra confirmed the PEGylation of the superparamagnetically doped polymer beads. Furthermore, the beads manifested a positive shift in their ζ-potentials after PEGylation, consistent with the loss of negative charges from the deprotonated free carboxyl groups. Electron microscopy showed that the PEGylation did not alter the morphology of the beads on micrometer and submicrometer scales (FIG. 12a,b), which was consistent with the formation of passivation layers with thicknesses that did not exceed a few tens of nanometers. Spectroscopic ellipsometry revealed that the thickness of the coatings did not increase proportionally with the length of the PEG chains. Furthermore, the extent of drying had a pronounced effect on the measured thickness of the PEG layers.

It is essential to consider the charged groups on the different substrates when submersed in the neutral-pH aqueous media: (1) the noncoated glass surfaces were negatively charged, (2) the noncoated polymer beads were also negatively charged due to their derivatization with carboxylates, (3) PEG-coated glass surfaces may possess residual positive charges buried in the PEG layers close to the glass surface because the PEG chains were grafted to aldehyde-functionalized glass via reductive amination leaving secondary amines at the PEG-glass interface, and (4) PEG-coated beads may possess residual negative charges due to nonreacted carboxylates (remaining buried under the PEG layers) after grafting the amine terminated PEGs to the carboxyl-functionalized beads via amide coupling. In addition, we prepared aminated glass slides for attaining a substrate with positively charged surfaces in which the charged groups were not buried under PEG coatings.

Noncoated beads with aminated glass surfaces provided attractive electrostatic interactions that were primarily derived from the negative charges from the deprotonated carboxyl groups on the beads and the positive charges from the protonated amines on the glass. In this experimental scenario, about 99% of the beads remained on the glass surface upon applying 1.2-pN force for five seconds. In contrast, noncoated beads and noncoated glass surfaces provided repulsive electrostatic interactions (where both surfaces were negatively charged), and only about 2% of the beads remained on the surface after the application of the magnetic force.

In certain embodiments of the invention, an image of a pathogen comprising image data is captured by a device and is automatically compared to a library of known pathogens using an image matching algorithm that may or may not include some or all of the following techniques: machine learning, artificial intelligence, Bayesian analysis, “fuzzy” logic.

In certain embodiments, the matching algorithm may either run native to the device itself or via an upload to a central data processing center for analysis. In certain embodiments, in the absence of a 100% probability match, a list of possible matches is provided along with probabilities. In certain embodiments, if a specific pathogen is not in the database a secondary analysis will attempt to match aspects of the pathogen to known families in the database. In certain embodiments, a device may be programmed to automatically upload HIPPA compliant data to relevant health authorities in the event of the detection of a dangerous/contagious pathogen.

### EXAMPLES

### Example 1

Pre-cleaned microscope glass slides (Corning; 75 9 50 mm; about 73% SiO2, 14% Na₂O, 7% CaO, 4% MgO, and 2% Al₂O₃) were purchased from VWR and cut into 25 9 8 mm pieces prior to use. Polished test grade silicon <100> wafers were purchased from Silicon Sense, Inc. and also cut into 25 9 8 mm pieces. 11-(Trietoxysilyl) undecanaldehyde acetal was purchased from Gelest, Inc. Indium (III) chloride (99.999%), redistilled N,N-Diisopropylethylamine (DIPEA) and sodium cyanoborohydride were all purchased from Aldrich. 3,6,9-Trioxaundecanedioic acid was obtained from Fluka. N,N-Diisopropylcarbodi-imide (DIC) and N-hydroxysuccinimide (HOSu) were obtained from Lancaster. Anhydrous hydroxybenzo-triazole (HOBt) was purchased from Chem-Impex International. Toluene, tetrahydrofuran (THF), and methanol, spectroscopy grade and anhydrous, were obtained from VWR and Fisher. Ethanol (dehydrated, 200 proof) was purchased from Pharmco. α,ξ-Diami-no polyethylene glycol (NH₂-PEG-NH₂) and methoxy amino polyethylene glycol (MeO-PEG-NH₂), MW=2000, were purchased from Nectar. Green fluorescent protein (GFP) was obtained from Upstate Signaling Solutions. Bovine carbonic anhydrase II (BCA) and 4-nitrophenyl acetate were obtained from Sigma.

### Surface Derivitization Procedures

The silicon wafers were cut and used without further cleaning with solutions. Prior to film preparation, the glass slides were cleaned by using ultrasonic treatment in toluene, dichloromethane, ethanol, ethanol-concentrated HCl (1:1, v/v), and rinsed with copious amounts of deionized water (MilliQ 18 X).

The surfaces of the glass and silicon substrates were blown dry with high-purity nitrogen and treated with oxygen plasma for 10 min prior the silanization. This treatment yields a hydrophilic surface with a native oxide layer.

### Silanization

Five oxygen-plasma treated substrate slides were immersed in 10 mL solution of 0.04% (v/v) of one of 11-(triethoxysilyl)undecanaldehyde acetal and 0.02% (v/v) N,N-diisopropylethylamine (DIPEA) in anhydrous toluene. The glass chambers used for this treatment offered a configuration that prevents the surfaces of the slides to touch one another or the walls of the chamber. The chamber with slides and silanization solution was heated on a sand bath and 1 h after the solution temperature reached 110° C., it was sonicated for 60. After overnight heating at 110° C., the slides were removed from the solution and doubly washed with toluene, dichloromethane, THF and ethanol (10 mL each), as well as with plenty of deionized water. The modified glass slides were kept immersed in deionized water and further derivatized within a two-day period.

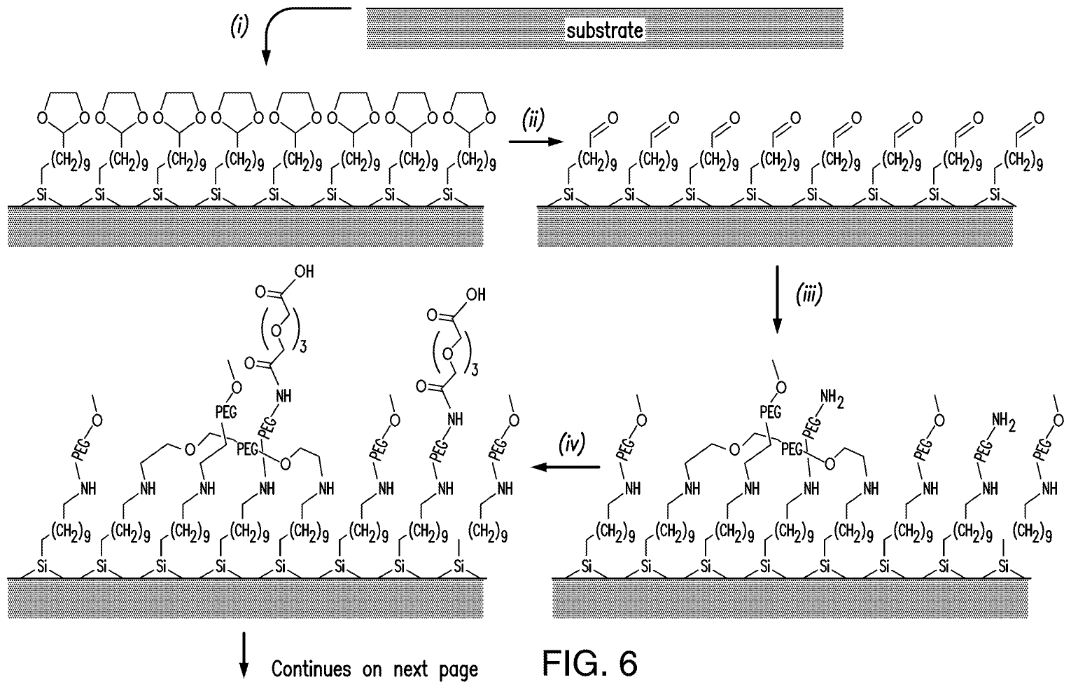

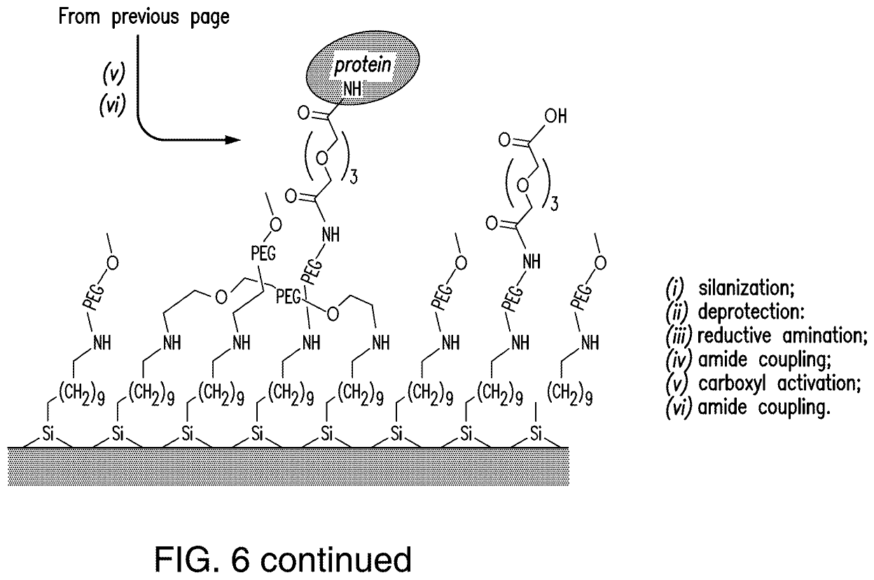

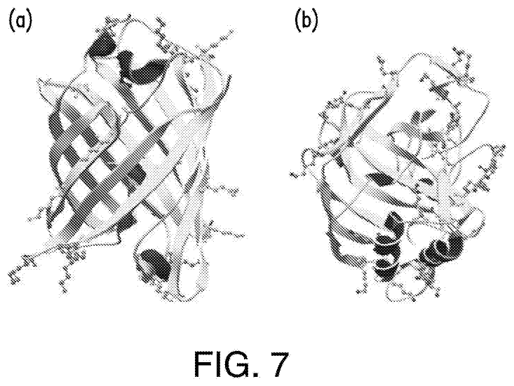

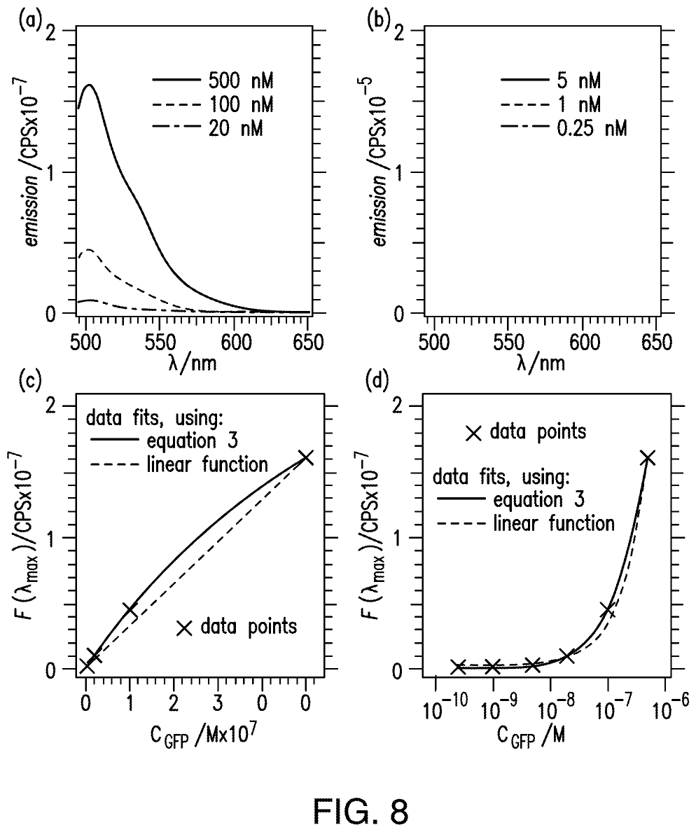

### Aldehyde Deprotection (See FIG. 6)

The acetal-coated substrates were taken out of deionized water, blown dry with ultrapure nitrogen and immersed in a 10 mL methanol and water solution (1:1, v/v) containing 2 mg InCl. The solution was heated to 80° C. After keeping it at this temperature for 1.5 h, the reaction mixture was allowed to cool down to room temperature and the substrate slides were taken out of the solution, washed and used immediately.

### Amine-Functionalized Polyethylene Glycol Layers (See FIG. 6)

Washed substrates with freshly deprotected aldehyde groups were blown dry and immediately immersed into a 10 mL methanol solution of diamino polyethylene glycol (NH2-PEG-NH2) RDM 9 30 mg (MW=2 kDa) and methoxy amino polyethylene glycol (MeO-PEG-NH2) (1-RDM) 9 30 mg (MW=2 kDa). The mixture was allowed to react for half an hour at room temperature and then sodium cyanoborohydride (20 mg, 30 mmol) was added to the solution. The mixture was shaken and reacted for additional 48 h at room temperature. The slides were removed from the solution and doubly washed with 10 mL methanol, THF, and 100 mL deionized water. Slides with RDM between 0 and 0.2 were prepared. All the modified substrates were stored in deionized water at 4° C. till use.

### Carboxylate-Functionalized Polyethylene Glycol Layers (See FIG. 6)

Substrates with amine functionalized PEG layers were blown dry and put into 10 mL dry THF with 3,6,9-trioxaundecanedioic acid (30 mg, 0.14 mmol), HOBt (25 mg, 0.18 mmol), DIC (0.1 mL), and DIPEA (0.05 mL). The mixture was shaken at room temperature overnight in the absence of light. The substrate slides were then removed and doubly washed with THF and deionized water.

### Covalent Attachment of Proteins to Polyethylene Glycol Layers (See FIG. 6)

Substrates with PEG layers functionalized with carboxylic acid groups were blown dry and placed in 10 mL dry THF solution of HOSu (20 mg, 0.17 mmol) and DIC (0.1 mL). The mixture was allowed to react at room temperature overnight in the absence of light. The activated substrates, i.e., the slides with the active OSu esters on their surfaces, were removed from the THF solution, doubly washed with dry THF, blown dry with nitrogen and immediately placed into a 10 mL PBS (phosphate-buffered saline) buffer (pH=6.85) solution containing 5 mg protein (GFP or BCA). The mixture was allowed to react for 24 h at room temperature at constant shaking. The slides were removed from the solution and washed with copious amounts of deionized water (MilliQ 18 MX) and PBS buffer. The modified substrate slides were kept in PBS buffer solution (pH=6.85) at 4° C. and used no later than one day after the preparation.

### Surface Characterization

The chemical composition of the surfaces was characterized with X-ray photoelectron spectroscopy and the thicknesses of the layers on the silicon substrates were determined using ellipsometry.

### X-Ray Photoelectron Spectroscopy (XPS)

X-ray photoelectron spectroscopy was performed with an SSX 100 ESCA spectrometer with mono-chromatized AlKa source (1486.6 eV). Survey spectra were collected from 0 to 1000 eV with pass energy of 188 eV, and high resolution spectra were collected for each element detected with pass energy of 23.5 eV. Survey and high-resolution spectra were collected at 65° take off angle, defined as the angle spanned by the electron path to the analyzer and the sample surface. All spectra were referenced by setting the carbon C1s peak to 285.0 eV to compensate for residual charging effects. The peaks as a shift of −1.5 eV and −3 eV from the C—C peak is the characteristic PEG coupling C—O peak and aldehyde C═O peak, respectively. Spectral analysis was performed using the software proved with the XPS instrument. Percents of atomic composition and atomic ratios were corrected using sensitivity factor incorporated in the software. The high-resolution C1s peaks were deconvoluted by a fit to a linear sum of Gaussians and a baseline correction.

Table 1 highlights the surface elemental composition extracted from analysis of survey spectra for glass surfaces coated with layers with various functionalities. Table 2 contains the results from the analysis of the C1s high-resolution spectra for the same substrates.

TABLE 1
Surface element composition for glass substrates
coated with layers with various functionalities.^{a}

| | % Si | % O | % C | % N |
|---|---|---|---|---|
| Blank | 24 | 70 | 5.4 | — |
| Acetal | 19 | 58 | 23 | — |
| Aldehyde | 14 | 64 | 22 | — |
| PEG | 14 | 50 | 32 | 4.0 |

^{a}Relative percentage (%) from survey spectra.

TABLE 2
Surface carbon composition for glass substrates
coated with layers with various functionalities.^{a}

| | % C—C | % C—O | % C═O^{b} |
|---|---|---|---|
| Blank | 100 | — | — |
| Acetal | 67 | 23 | 9.8 |
| Aldehyde | 70 | 15 | 16 |
| PEG | 66 | 32 | 2.2 |

^{a}Relative percentage (%) from high-resolution C1s spectra.
^{b}The C1s binding energy for C═O and O—C—O are indiscernible.

The decrease in the relative amount of C—O, accompanied with an increase in the relative amount of C═O, upon deprotection of the aldehydes (i.e., transformation of acetals to aldehydes) is indicative for the chemical removal of the ethylene glycol protection group in the acetal. The appearance of nitrogen peak and the increase in the relative amount of C—O, with concurrent decrease in the relative amount of C═O, are indication for the successful coupling of the PEG to the aldehyde surface via reductive amination. The chemical surface reaction conducted on silicon substrates showed identical trend.

### Ellipsometry

Ellipsometric measurements, conducted in air, were performed at 44 wavelengths (2) between 400 and 700 nm and at three angles of incidence (0) using M-44 spectroscopic ellipsometer, J. A. Woollam Co., Inc. The phase (D), and the amplitude (W) were determined with uncertainty of less than 0.005°, 0.02°, and 0.01°, respectively, for three different angles, 0=60°, 70°, and 80°. The complex refractive index (n=n+ik) and layer thickness (d) were obtained from fits of D and W vs. 2 and 0, using a Newton-Raphson solution of Fresnel's reflectivity equations for multilayer systems.

For the wavelength range of the measurements, the absorptive contributions embodied in n were negligibly small for all the layers and k was set to zero for the models. For the wavelength-dependent values of n for some of the materials, we used a three-term Cauchy model (C in Eq. 1 is not related to concentration):

$$n ( λ ) = A + \frac{B}{{λ}^{2}} + \frac{C}{{λ}^{4}}$$

For bare silicon wafer, treated with oxygen plasma, the known values of n(k) for SiO2 were used. For the undecylaldehyde acetal layers, we used the published values of the wavelength dependent refractive indexes for alkanes for initial guesses for A, B, and C. For polyethylene glycol, the reported values of B and C are significantly smaller than A, making the nonlinear terms (i.e., second and third term) in Eq. (1) statistically insignificant for the data fits for substrates with PEG layers.

Ellipsometry measurements for acetal-derivatized silicon revealed that upon treatment with oxygen plasma, the silicon wafers were covered with about 2 nm layer of silicon dioxide. The data from the silanized substrates could not be fit to a single-layer model. A two-layer model, encompassing a layer of SiO₂ and a layer of the alkyl material over it, yielded a thickness of 1.0 (±0.3) nm for the undecylaldehyde acetal layer.

For PEG-derivatized surfaces, the ellipsometry data indicated the average thickness of the polymer layers is 11 (±0.2) nm. Based on reported PEG density (1.1-1.2 g/cm³), and the molecular weight of the macromolecules (MWPEG=2 kDa), the surface concentration was calculated to be 3.5 (±0.6) PEG molecules per nm².

The true coated areas, therefore, are larger than what is considered in the estimation, leading to overestimation of the surface density. The ellipsometry measurements are performed on dry surfaces: i.e., the PEGylated substrates are taken out of the aqueous environment, dried with a stream pure

### Small-Angle Emission Measurements

The emission spectra of GFP were recorded using a spectrofluorometer, Fluorolog-3-22, equipped with a 21-degree-angle collection adapter for surface-emission experiments. GFP-derivatized glass slides, with RDM between 0 and 0.2, were immersed in a cuvette filled with 3 mL PBS buffer, pH 6.85, and their emission and excitation spectra were recorded. For calibration, the fluorescence of PBS-buffered solutions of GFP with concentrations between 0 and 500 nM were recorded under identical settings of the spectrofluorometer. The fluorescence intensities, F, measured for the calibration solutions with different concentrations of GFP were fitted to Eq. (3). The obtained parameter FGFP were input in Eq. (4) for calculation of the GFP surface concentration.

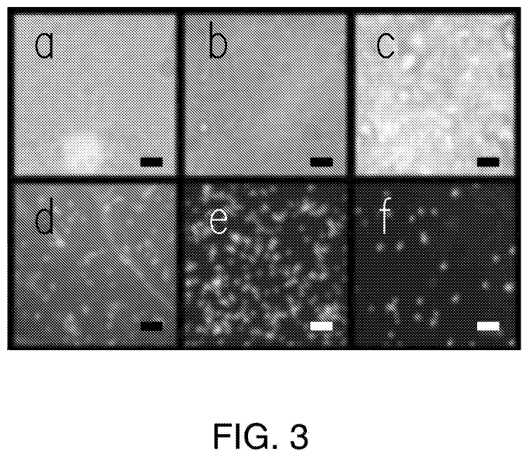

From the emission intensities, FS, recorded for GFP immobilized on glass slides with different RDM, the corresponding surface concentrations of the protein, CGFP (in molecules/nm²) were calculated using Eq. (4) (FIG. 3). Each set of solution and surface measurements were collected with identical settings of the spectrofluorometer within 3-6 h period. The obtained surface concentrations, CGFP, were plotted against RDM and fitted to a sigmoidal function (FIG. 3b).

### Kinetic Measurements

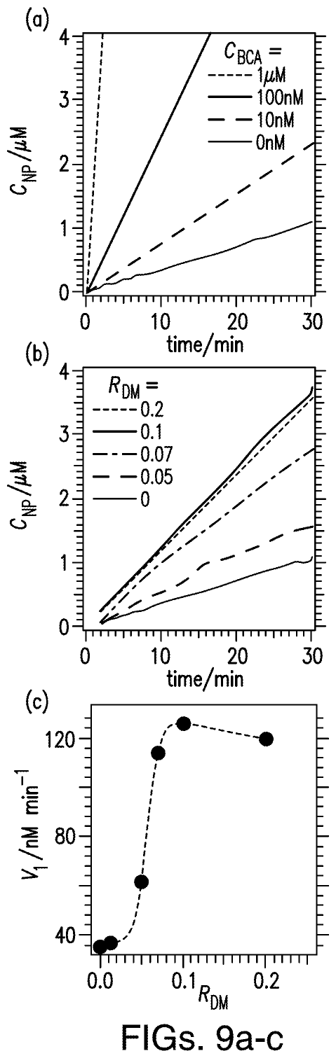

The kinetic assays for the BCA enzyme were carried with a Varian Cary (scanning wavelength) UV/Visible spectrophotometer. For homogeneous catalysis, the cuvette was filled with 3-mL BCA solutions with different concentration, between 0 and 10 lM. For heterogeneous catalysis, glass slides with BCA immobilized on them (RDM between 0 and 0.2) were placed in a cuvette filled with three milliliters aqueous HEPES buffer, pH=8.25. At time zero of the measurement, 30 IL of 10 mM solution of NPA was added and the solution was quickly stirred. The change in the absorption at 348 nm was recorded for 30 min. The background absorbance at time zero was subtracted from the data and the kinetic curves were divided by 5000 M¹ cm¹, which is the molar extinction coefficient of both, nitrophenol and nitrophenolate, at 348 nm. From data fits of CNP vs. time, the rate of formation of the product, nitrophenol(ate), NP, was extrapolated to time zero. The obtained initial rates, (i.e., the rates at time zero), were plotted against RDM and fitted to a sigmoidal function. (See FIG. 9a-c).

### Surface Derivatization

Using identical surface-chemistry procedures for glass and silicon substrates, we prepared a series of interfaces and investigated their properties. Due to the insufficient reflectivity of the glass, we conducted ellipsometry measurements only with the silicon samples. Because of the opacity of the silicon, UV/visible absorption and emission data were recorded solely for the glass-supported interfaces.

Although the two substrates, silicon and glass, have very different bulk properties, their surface composition can support similar types of chemistry. Silicon dioxide accounts for more than 70% of the content of the glass. The oxygen-plasma activation of the substrates, applied prior to the silanization, generates a thin layer (˜2 nm) of silicon dioxide on the silicon surfaces. Therefore, the discussed surface derivatization involves attachment of bioactive layers to silica, rather than silicon, interfaces.

### Considerations in the Strategy for Surface Derivatization

Coating the surfaces with bioinert layers, prior to protein attachment, is essential step in the preparation of bioactive interfaces. Bioinert coatings provide: (1) resistance against nonspecific interactions; and (2) prevention of denaturation (and loss of activity) of the surface-bound proteins.

Polyethylene glycol (PEG) is the preferred polymer for constructing bioinert interfaces. The properties of polyethylene glycol make it unique among other water-soluble polymers. A small Flory-Huggins parameter and close-to-unity intramolecular expansion factor characterize the interactions of PEG with water. The amphipathic composition of this polyether, containing hydrophobic ethylene units (—CH₂—CH₂—) and hydrogen-bond-accepting ether oxygens (—O—), appears to dictate its behavior and conformation in aqueous media. Other polyethers, such as polymethylene oxide (i.e., polyformaldehyde) and polypropylene oxide, for example, with higher or lower relative content of ether oxygens, respectively, are not water soluble.

Covalent attachment of polymers to silica-based surfaces can be performed in a single-step process (i.e., direct covalent attachment of the polymer to the substrate), or via multiple-step procedures. While the single-step approaches for derivatization of surfaces are expedient, they are limited by the availability of polymer derivatives containing functional groups, such as alkoxysilyls, necessary for anchoring the macro-molecules to the surfaces. Furthermore, solubility and conformational issues related to the polymer derivatives may limit the choice of media and conditions for conducting the single-step processes.

Alternatively, multiple-step approaches for derivatization of silica-based surfaces allow for significant flexibility and diversity in the types of coatings that can be prepared. Initial silanization of surfaces with small x-functionalized (trialkoxysilyl)alkanes, under optimal conditions for this particular reaction, can produce densely packed SAMs with functionality for covalent attachment of polymers onto them.

Two different approaches can be undertaken for preparation of polymer layers that are covalently attached to functionalized SAM surfaces: (1) polymerization at the SAM interface by introduction of monomers or prepolymers and initiation of the polymerization with the SAM functional groups; and (2) covalent attachment of already synthesized polymers to the SAM functional groups. The second approach allows for considerably better control of the thickness and functionality of the formed coatings.

Not all high-yield solution-phase reactions are appropriate for surface derivatization. Reactions that proceed through bulky intermediates are hindered when forced to undergo at high-packing density onto heterogeneous interfaces. The immensely wide used carbodiimide chemistry for amide coupling, for example, should be employed with caution in surface-derivatization procedures due to the relatively large size of the active-ester intermediates. Therefore, we utilized silanization and reductive amination (both proceeding through intermediates with relatively small sizes) for achieving well packed PEGylated surfaces. The consequent steps of anchoring the proteins to the relatively flexible PEG chains at “diluted” surface concentrations do not have such steric restrictions. Carbodiimide chemistry was employed for the last two chemical transformations.

A multiple-step approach was undertaken for chemical derivatization of silica-based surfaces. Each step was conducted under optimal conditions and in the most appropriate media for each particular reaction. We directly attached PEGs with molecular weight of about 2 kDa to aldehyde-coated substrates, allowing amine functionality on the PEG layers. Conversion of amine to carboxyl functionality allowed for covalent attachment of proteins to the PEGylated surfaces via amide coupling.

Silanization

Due to their reactivity, aliphatic aldehydes present an excellent choice for preparation of chemically active surfaces. Their reactivity, however, can compromise the quality and durability of the formed active SAMs. Therefore, we chose to functionalize the substrates with acetals, i.e., protected aldehydes.

To suppress extensive crosslinking between the silyl conjugates in the SAMs, we silanized glass and silicon substrates under anhydrous conditions at elevated temperature for extended time periods: i.e., toluene, −110° C., −12 h. (Such crosslinking contributes to the formation of multilayers and defects in the surface coatings.) Furthermore, for obtaining coatings with improved quality, we chose alkoxysilyl derivatives as silanization agents, instead of the more reactive halo-silyls. We observed, for example, that silanization with trichlorosilyls results in the formation of micrometer-size multilayer aggregates that significantly increase the roughness of the surfaces. In comparison, identical silanization with trimethoxy- or triethoxysilyls yields lesser amount of smaller-size aggregates and hence, produces interfaces with smoother topography.

In addition, we employed short mechanical treatments of the substrates within the first one-to-two hours of silanization. Examination with atomic force microscopy (AFM) and reflection interference contrast microscopy (RICM) showed that sonication for one minute at one hour after the initiation of the silanization, resulted in surface coatings with significantly decreased number of defects.

The employed method under the discussed conditions of silanization with 11-(triethoxysilyl)undecanal-dehyde acetal produced one-nanometers-thick coatings with acetal functionality.

Aldehyde Deprotection

Conversion of acetals to aldehydes is typically carried in strong acidic media. Strong mineral acids, however, react with glass surfaces and hence, can wash away the SAMs. Moderately weak Lewis acids can catalyze acetal hydrolysis under relatively mild conditions. Adopting such an approach for aldehyde deprotection, allowed us to activate the acetal-coated substrates by converting the acetals to aldehydes using indium (III) ions, rather than mineral acid, as a catalyst. This treatment of the acetal-coated surfaces resulted in: (1) an increase in the relative content of the carbonyl carbons, as measured with XPS; and (2) a decrease in the wetting contact angle (with water) from 81°±6° to 55°±14°. Both changes are consistent with the removal of the ethylene groups from the surface coating. We always conducted the surface activation, i.e., aldehyde deprotection, immediately before the next derivatization step.

PEGylation

Due to its simplicity and high yields, reductive amination is broadly used for anchoring of macromolecules to interfaces. Reductive amination occurs spontaneously at room temperature. Water, alcohols and other oxygen nucleophiles, typical for biological fluids, do not compromise the yields of reductive amination.

This reaction between aldehydes and primary amines proceeds through small-size intermediates leading to the formations of imines. This initial amination step is reversible: i.e., the imines are readily hydrolysable. Reduction with hydride agents converts the imines to the final secondary amines that provide a stable carbon-nitrogen-carbon covalent linkage. Overall, the reductive amination proceeds through intermediates that do not impose steric hindrance, and hence, it is appropriate for conducting high-yield coupling reactions at interfaces and for pursuing high-density packing.

For reducing agent, which converts the imines to the final amines making the coupling irreversible and unsusceptible to hydrolysis, we chose a hydride donor with moderate strength, sodium cyanoborohydride, to avoid possible side reactions and heating of the reaction mixture.

Covalent attachment of amine derivatives of PEGs (MW ˜2 kDa) to freshly deprotected aldehyde sur-faces, via reductive amination, yielded polymer layers containing about three PEG molecules per nm². Using mixtures of monoamine and a,x-diamine polymers, H₂N-PEG-O—CH₃ and H₂N-PEG-NH₂, respectively, allowed us to prepare PEG layers with free amines on their surfaces.

We used the molar fraction of the diamine in the reaction mixture, RDM, as a semi-quantitative indication of the expected amount of free amine groups on the surface:

$${R}_{DM} = \frac{{C}_{{H}_{2} N} \_PEG {\_NH}_{2}}{{C}_{{H}_{2} N} \_PEG {\_NH}_{2} + {C}_{{H}_{2} N} \_PEG \_O {\_CH}_{3}}$$

where CH₂N_PEG_NH₂ and CH₂N_PEG_O_CH₃ are the concentrations of the PEG diamine and monoamine, respectively, in the reductive amination reaction mixtures.

For the surface molar fraction of the primary amines, vNH₂; it can be assumed that vNH2 2RDM for RDM <<1 if: (1) all diamine polymer chains are anchored to the substrate via only one of their termini and (2) the amine groups at the termini of the mono- and diamine polymers have identical reactivities. While increase in the length of the polymers should increase the plausibility of the latter condition, the flexibility of the PEG chains compromises the plausibility of the former condition. (The flexibility of the PEG chains increases the probability for the distal amine of a singly attached bifunctional polymer to come in contact with the interface.) Therefore, we expect vNH2<2RDM.

We used different reaction ratios, RDM, for coating aldehyde-activated surfaces. The primary amines at the PEG interface, however, do not provide means for direct covalent coupling to proteins without encountering undesirable side reactions. Therefore, we converted the amine functionality of the PEGylates substrates into carboxyl functionality by treatment of the substrates with preactivated 3,6,9-trioxaundecanedioic acid (Scheme 1, iv).

Biofunctionalization

Using amide coupling allowed for covalent attachment of proteins to the PEGylated interfaces. We activated the carboxylates at the PEG interfaces under anhydrous conditions (Scheme 1, v). Immediately after the activation, the substrates were transferred into an aqueous solution of the protein of interests, buffered at optimal pH (Scheme 1, vi).

Such an amide-coupling method for protein attachment presents two principal disadvantages: (1) depletion of positive surface charges of the protein (due to conversion of amines to amides) may change its functionality; (2) indiscriminate coupling to any of the amines (from surface lysines or N-termini) will result in random orientations of the proteins at the PEGylated interfaces. Decrease in the surface concentration of the functional groups at the PEG interfaces will decrease the severity of the second issue. In fact, decrease in the surface concentration will assure that each protein molecule binds to the surface through only a single covalent bond, depleting only a single positive charge per a molecule. Furthermore, decrease in the surface concentration and increase in the length of the PEG linkers, which connect the biomolecules to the interface, will allow relatively free rotational diffusion of the proteins making the randomness of their binding to the substrate less of an issue.

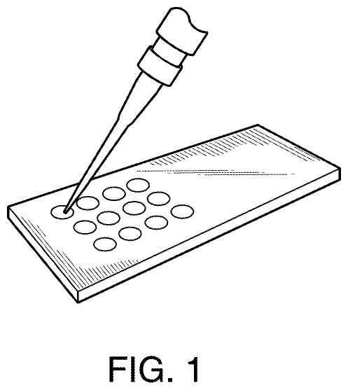

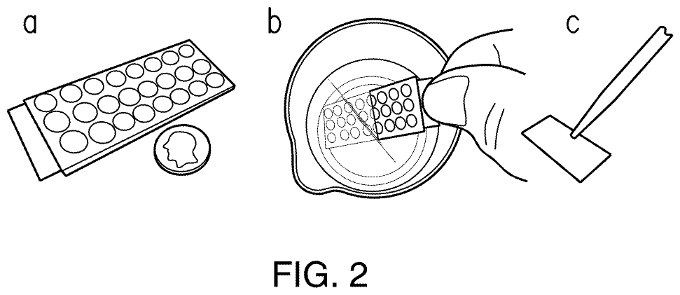

Substrates with a broad range of surface concentrations were prepared to examine if the functionalities of the interface-bound proteins are compromised. As a proof of principle, we immobilized two proteins with molecular weights of about 30 kDa onto PEGylated glass and silicon substrates (FIG. 1). Small-angle emission spectroscopy allowed us to estimate the surface concentrations of green fluorescence protein (GFP) immobilized to the PEG interfaces prepared at various RDM ratios. Alternatively, a surface-bound enzyme, bovine carbonic anhydrase II (BCA), exhibited activity catalyzing ester hydrolysis, which we observed using absorption spectroscopy.

Small-Angle Fluorescence Studies

Small-angle fluorescence spectroscopy allowed us to determine the surface concentration of GFP, FGFP, attached to PEGylated glass substrates. We used fluorescence spectra of solutions of GFP to generate the calibration curves required for the concentration measurements. The fluorescence intensities, F, measured for the calibration solutions with different concentrations of GFP were fitted to:

F=F₀(1−10^{-aC}^{_{GFP}})

where F0 depends on the fluorescence quantum yield of GFP and the properties of the spectrometer; CGFP is the solution concentration of GFP; and is the product of the molar extinction coefficient of GFP at the excitation wavelength and the excitation pathlength.

The dependence of the fluorescence intensity on the fluorophore concentration is described as a liner relationship. Such linear representation, however, is an approximation. The failure of this approximation becomes particularly conspicuous for data with a broad dynamic range: i.e., for concentration ranges spanning a few orders of magnitude. Therefore, we chose to use the exact expression for F vs. C, as shown on Eq. (3), that produced superior data fits in comparison with the linear approximation.

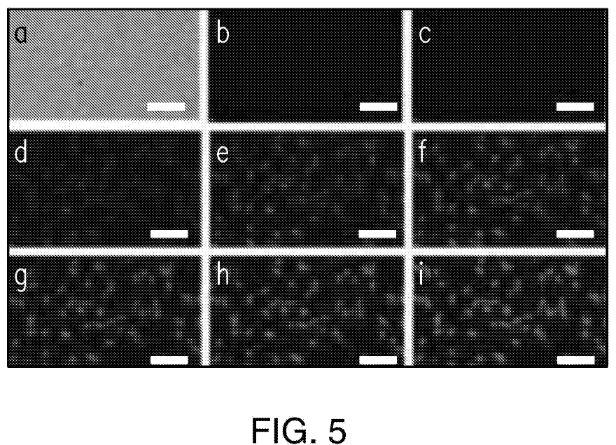

The similarities in the emission spectra of the sur-face-bound protein and the protein solutions indicate that a covalent attachment of GFP to PEGylated interfaces does not compromise its structural integrity and alter the microenvironment of its fluorophore (FIG. 5). From the emission intensities, FS, recorded for GFP immobilized on glass slides with different RDM (FIG. 3), we calculated the corresponding surface concentrations of the protein, FGFP, using the calibration parameters F0 and x (Eq. 3):

$${Γ}_{GFP} = \frac{{N}_{A}}{\text{?} 2 \times {10}^{17}} log ( \frac{{F}_{0}}{{F}_{0} - {F}_{S}} )$$

$$\text{?} \text{indicates text missing or illegible when filed}$$

where NA is the Avogadro's number, and the conversion factor, 2 9 1017, yields FGFP in molecules per nm2 if CGFP in Eq. (3) is in moles per liter. The factor “2” in the denominator is introduced for transparent substrates, in which the front and the back surface are illuminated and both interfaces are sources of emission.

At small RDM, FGFP increases relatively slowly with the increase in RDM. Increase in RDM above 0.05 causes a rapid increase in FGFP with a rate, FGFP/RDM, of about two molecules per nm2. For RDM exceeding 0.1, FGFP plateaus at maximum value of about 0.13 molecules per nm2, which corresponds to an average surface area of about eight nm2 for a single GFP molecule.

Considering its dimensions, a single GFP molecule can cover an area between 7 and 15 nm², corresponding to 0.07-0.14 molecules per nm2. (GFP can be approximated to a cylinder with a diameter of about 3 nm and height of about 5 nm Our findings suggest that at RDM∀0.1, GFP forms a tightly packed layer.

Kinetic Assays

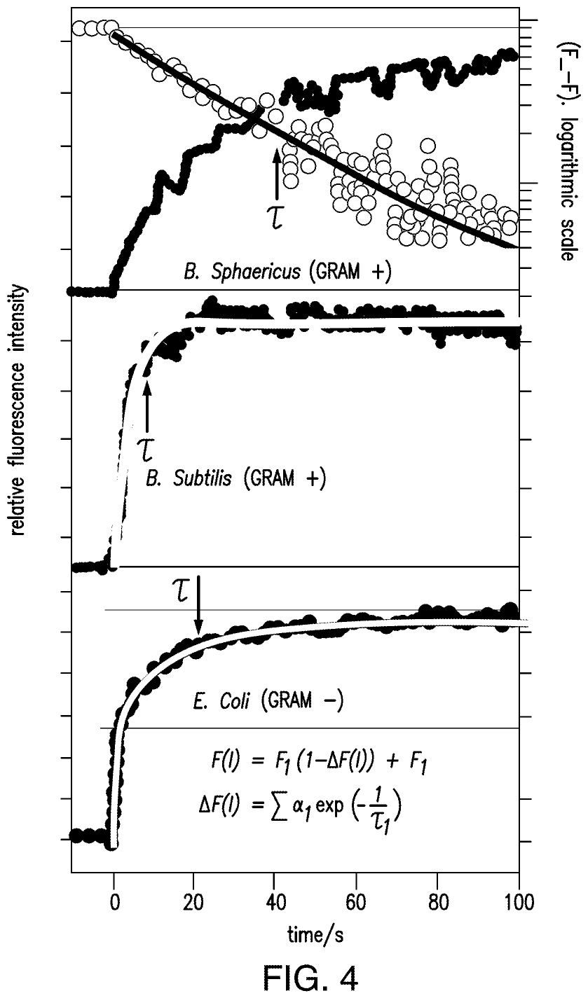

Carbonic anhydrase is a zinc-containing metaloprotein that catalyzes the reversible conversion of carbon dioxide to carbonic acid (existing predominantly in the form of bicarbonate under physiological conditions). Carbonic anhydrase is also known to catalyze the hydrolysis of various aromatic esters. These processes occur at the same active site where the hydration of CO2 is catalyzed. We monitored the hydrolysis of 4-nitrophenyl acetate (NPA) in the presence and absence of BCA. The hydrolysis of NPA produces 4-nitrophenol (NP) and acetic acid. For the pH range, in which BCA is active, the nitrophenol is in equilibrium with nitrophenolate. Using UV/visible absorption spectroscopy, we monitored the production of NP at 348 nm—a wavelength at which the protonated and deprotonated forms of NP have the same molar absorption extinction coefficients, e=5.0 9 103 M¹ cm¹ (FIGS. 4a and 4b).81

For homogeneous biocatalysis measurements, an increase in the solution concentration of BCA increased the rates of hydrolysis of NPA (FIG. 4a). For heterogeneous biocatalysis measurements, we immersed PEG-coated glass slides, with various amounts of BCA immobilized on their surfaces, into aqueous solutions of NPA. Indeed, we observed faster rates of hydrolysis when the glass slides were derivatized with higher surface concentration of BCA.

When the amount of substrate significantly exceeds the amount of enzyme, the initial rate of the catalyzed reaction, vi, is linearly proportional to the concentration of the active enzyme. Comparison between the initial rates recorded for homogeneous and heterogeneous catalysis allowed us to estimate that the value of vi observed for CBCA=10 nM (FIG. 4a) is in the range of the values of vi for RDM between 0.05 and 0.07 (FIG. 4b). A consideration of the volumes of the samples and the areas of the coated surfaces, allows for estimations that indicate that enzyme volume concentration, CBCA=10 nM, corresponds to enzyme surface concentration, FBCA=0.04 molecules per nm². This estimation suggests that FBCA 0.08 molecule nm² for the maximum rate of heterogeneous hydrolysis observed at RDM=0.1 (FIG. 4c).

Despite their apparent plausibility (i.e., FBCA FGFP for identical RDM), the results from such estimation should be taken more like qualitative guidelines. A calculation of FBCA from CBCA, based on enzymatic reaction rates, will result in underestimation of FBCA because of the constraints inherent for the heterogeneous reactions: e.g., limits in enzyme diffusion and

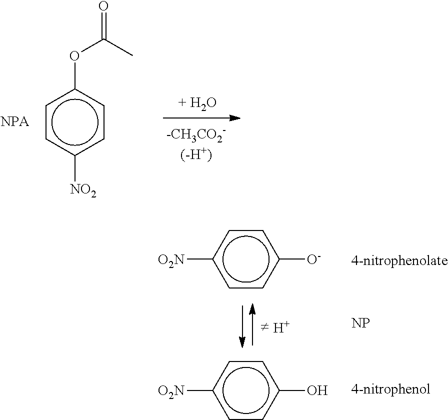

mass transport at the bioactive interfaces. Nevertheless, the estimated values for FBCA, which are expected to be underestimated, are slightly smaller than the values for FGFP at the same RDM (£0.1). Because the surface concentrations of BCA were calculated from comparison between solution and heterogeneous kinetics, our findings suggest that the surface-bound BCA preserves its functionality for catalyzing ester hydrolysis.

For RDM <˜0.1, the dependence vi on RDM showed a trend quite similar to the trend observed for the dependence of FGFP on RDM (FIGS. 3b and 4c). Unlike GFP, however, the activity of surface-bound BCA reaches a maximum at RDM 0.1 and consequently shows a decrease at higher values of RDM (FIG. 4c). Two different phenomena may contribute to the observed decline in BCA activity at RDM exceeding 0.1:

(1) The protein molecules are connected to the PEG interface via their surface amines and some of the lysine residues are in the proximity of the active site. Therefore, tight packing of the proteins on the surface, achieved at RDM 0.1, may limit the accessibility to some of the active sites Similar steric constraint may result from the formation of multiple covalent bonds between a single protein molecule and the interface at large RDM values. Such multiple binding will impede the rotational diffusion of the enzyme molecules preventing an efficient exposure of their active sites to the solution.

(2) Acetic acid is a weak competitive inhibitor for carbonic anhydrase. Increase in the surface density of the enzyme may not allow an efficient diffusion of the produced acetate (Scheme 2) away from the interface. The observed decrease in BCA activity for relatively large RDM values could be a reflection of partial inhibition resultant from increase in the local acetate concentration.

Dependence of Protein Surface Concentration on RDM

For GFP and BCA, the dependence of the protein surface concentration, Fp, on the reaction ratio, RDM, shows three distinct regions (FIGS. 3b and 4c): (1) a slow increase in Fp with RDM at RDM <0.05; (2) a sharp increase in Fp with RDM at 0.05<RDM <0.1; and (3) surface saturation, where Fp reaches the maximum at RDM>0.1.

At RDM 0.1, maximum surface packing of the proteins is achieved. As expected, further increase in the surface concentration of carboxyl groups on the PEG layers will not result in increase in the protein surface concentration due to steric hindrance. The reasons for the nonlinear dependence of Cp on RDM, at RDM <0.1, resulting in two distinct regions, however, are not as obvious. The observed nonlinearity indicates that an increase in RDM causes: (1) a nonlinear increase in the surface concentration of functional groups on the PEG layers; and/or (2) a nonlinear increase in the propensity for coupling of the proteins to the functionalized PEG layers.

In the described procedure, either one or both termini of the a,x-diamines and dicarboxylates can attach to the surface layers (Scheme 1, iii and iv). The observed nonlinear dependence of protein surface concentration on RDM, therefore, could suggest that an increase in the molar ratio of the bifunctional conjugates in the reaction mixture, i.e., an increase in RDM, results in an increase in the portion of bifunctional conjugates that are surface-bound only through one of their termini.

A possible increase in the protein adsorptivity of the PEG interfaces with the increase in the amount of surface functional groups presents an alternative reason for the observed nonlinearity of Cp vs. RDM. Through the conversion of the PEG functionality to carboxyl, all primary amines on the PEG interface are transformed into amides (Scheme 1, iv), which may increase the surface adsorptivity for proteins. Furthermore, prior to the protein coupling, the carboxylic acid on the PEG surfaces is converted into an active ester, which is more hydrophobic than the carboxyl and methoxy groups. The increase in the surface fraction of amides and esters can lead to increase in the efficiency of the protein physisorption, which is the initial step of the chemical coupling reaction.

It is unlikely for electrostatic forces between the proteins and the deactivated carboxylates, which are negatively charged, to play a role in the increase in the physisorption propensity with the increase in RDM because the isoelectric points of GFP and BCA have smaller values than the pHs of the buffers used for the coupling reactions.

### Example 2

Materials. Polymer microspheres, ProMag, superparamagnetically doped with magnetite (that have carboxyl functional groups, with a mean diameter of 3 sm and 1.9 g/cm3 density) were purchased from Bangs Laboratories, Inc., in January 2009. Precleaned 1 mm thick microscope glass slides were purchased from VWR and cut into 25×35 mm pieces prior to use. Silicon wafers (n/phosphorus and p/boron doped, 1-10 Ωcm, one side polished, test grade, (100)) were purchased from Silicon Sense, Inc., and cut into 25×10 mm prior to use.

The polymers, α,ω-aminomethoxy polyethylene glycol (MeO-PEG-NH2), MW=1, 2, 3, 5, 10, and 20 kDa, were purchased from Layson Bio. 11-Aminoundecyltriethoxysilane and 11-(trietoxysilyl)-undecanaldehyde acetal were purchased from Gelest, Inc. Indium(III) chloride (99.999%), redistilled N,N-diisopropylethylamine (DIPEA), TWEEN20 surfactant, and sodium cyanoborohydride were purchased from Aldrich. N,N-Diisopropyl-carbodiimide (DIC) and N-hydroxysuccinimide (HOSu) were obtained from Lancaster. Hydroxybenzo-triazole (HOBt) was purchased from Chem-Impex International. 1-Ethyl-3-(3-dimethylaminopropyl) carbodiimide (EDC), toluene, tetrahydrofuran (THF), methanol, and ethanol, all spectroscopy grade and/or anhydrous, were obtained from Fisher.

Coating and Characterization of Flat Substrates. Using a surface chemistry protocol that we previously developed, we functionalized glass slides with varying lengths of MeO-PEG-NH₂. For ellipsometry measurements, we prepared identical coatings on silicon wafers. We monitored the completion of PEGylation reaction via surface contact angle measurements.

For the aminated control surfaces, we salinized glass slides with alkyleneamines by (1) cleaning them as we previously described, (2) treating them with oxygen plasma, and (3) immersing them in an anhydrous toluene solution of 0.04% (v/v) of 11-aminoundecyl-triethoxysilane and 0.02% DIPEA. The glass slides, immersed in the salanization solution, were heated on a sand bath at 110° C., and were sonicated in hot water intermittently for 60 s after the first hour. After 18 h, the salanization bath was allowed to cool to room temperature, the solution was discarded, and the glass slides were doubly washed with toluene, dichloromethane, THF, ethanol, and deionized/milli Q water.

Coating and Characterization of Microbeads. The PEGylation of the beads involved the following principal steps.

Cleaning. Ten sL of a suspension of magnetic beads were placed in a 1 mL microfuge tube containing 250 sL solution of 0.01 M sodium hydroxide (NaOH), and gently shaken at room temperature for 10 min. The beads were trapped with a permanent magnet at the bottom of the tube for 5 min and the solution was decanted. This wash step was repeated twice. 500 sL of deionized water was then added to the centrifuge tube and gently shaken at room temperature for 5 min. The water-bead solution was decanted and the process repeated three times.

PEG Coupling. In a cold room, the water was removed from the centrifuge tube, and 200 sL solutions of HOSu and EDC were added to the beads, followed by addition of MeO-PEH-NH₂. The solution was gently shaken in the cold room (4° C.). After overnight shaking, the solution was brought back to room temperature for 2-4 h and washed with plenty of deionized water and decanted. Beads were stored in deionized water at 2-8° C. until needed.

Verification. The presence of the PEG coatings was confirmed using FTIR as we have previously demonstrated. The completion of the coupling reaction was monitored by measuring the c-potential of the beads. The covalent modification of the polymer beads with PEG led to a positive shift in the c-potentials, resultant from the elimination of the negatively charged carboxylic acid groups.

Ellipsometry. Spectra of the phase (Δ) and the amplitude (Ψ) were recorded between 400 and 700 nm for three angles of incidence, 60°, 70°, and 80°, using Horiba Jobin Yvon UVISEL spectroscopic ellipsometer, model M200. The measurements were conducted in a class-1,000 clean room, with humidity maintained at 45 (±1) %. The samples were mounted on an ellipsometer stage exposed to air.

For the “wet” PEG coatings, the samples (coated silicon wafers stored under water) were washed with Milli-Q water and kept vertically to dry over dust-free wiper tissue for at least half an hour prior to the measurements. For the ellipsometry spectra of the “dry” coatings, the PEGylated surfaces of the same samples were blown with copious amounts of dry nitrogen for at least 5 min.

The thicknesses of the PEG layers, hdry and hwet (Table 1), were obtained from global fits of the ellipsometry spectra recorded at the different angles. A two-layer model, air/PEG/SiO2/Si, provided excellent fits for the spectra of all samples. Using models based on a single layer or on more than two layers did not yield satisfactory data fits. The fitting algorithm minimized the values and the fitting residuals revealed the goodness of the fits. The thickness of the SiO2 layer was about 2.2 nm and the thickness of the PEG layer varied with the MW of the polymer.

Established parameters for all materials were incorporated in the data analysis software, DeltaPsi. For the PEG material layer in the fitting model, we used the default parameters provided by the Thin Film Division at HORIBA Jobin Yvon, Inc., ∞=1.0, s=1.633, wt=9.723, and Γ0=4.921.56 For this study, we used the layer thicknesses obtained from data fits for which these parameters were fixed. Relaxing these parameters for the analyses of the “dry” samples did not improve the quality of the fits, and produced negative values for some of the dielectric quantities. Relaxing the parameters for the “wet” samples resulted in a slight improvement of the quality of the fits, and, within physically feasible values of the quantities characteristic of the PEG material, yielded layer thicknesses of 2.7, 9.1, 22, 35, and 27 nm for PEGs with MW 1, 2, 3, 5, 10, and 20 kDa, respectively. Nevertheless, because we did not have a basis for judging the plausibility in the variations of the fitting dielectric and optical parameters, we used the results from the data fits with fixed parameters (i.e., using a model for pure PEG for analyzing the data from the wet PEG samples).

Microscopy. Fluorescence microscopy images were acquired using a Nikon Ti-U inverted microscope (Nikon, Inc., Melville, N.Y.), equipped with a 100× Nikon oil immersion objective (numerical aperture, 1.49; working distance, 120 μm) and a Hamamatsu electron multiplier charge-coupled-device digital camera (model C9100-13; Hamamatsu Corp., Bridgewater, N.J.), as we previously described. Suspension of superparamagnetic beads was dropped on a glass slide, placed over the objective of the microscope, and the beads were allowed to settle on the glass surface. Using bright-field mode, the objective was focused on the settled beads, and for the imaging, it was turned into fluorescence mode utilizing the autofluorescence from the polymer material composing the microspheres (λem=536 nm, bandwidth=25 nm).

Scanning electron microscopy images were recorded using FEI XL30-FEG SEM. Prior to imaging, the superparamagnetic beads were washed with Milli Q water several times, lyophilized, spread on a sample stage, and sputter-coated with a conductive layer (80% Pt and 20% Pd).

Calibration of the Magnetic Puller. We built a magnetic puller setup to carry out the adhesion studies. We designed the setup in two interchangeable configurations: (1) “work mode” for measurements of the number of beads on a flat surface of a transparent substrate (Scheme 1a), in which the objective is positioned below the sample slide, and (2) “calibration mode” for force calibration (Scheme 2), in which the objective provides a side view of the suspension allowing for tracking the position of beads as they move vertically in response to the magnetic field. The two parameters used for controlling the force experienced by the paramagnetic beads are (1) the distance from the core of the electromagnet and (2) the current flowing through the coil of the electromagnet, controlled by the applied voltage.

Stoke's drag equation allowed us to determine the forces generated on each super paramagnetic bead at different distances and different applied voltages (FIG. 5)

Fd=−6πμrv

where Fd is the drag force, μ is the dynamic viscosity of the media, and r is the radius of a bead that moves with velocity v. The viscosity of the solutions used for the force calibrations was measured using a Cannon-Fenske N 956 Size 150 viscometer.

Because the employed forces were relatively weak, we accounted for the gravitational pull on the suspended beads. The magnetic force that pulls the beads upward opposes the drag force and the gravitational force. Two different approaches allowed us to determine the gravitational force: (1) calculate it by accounting for the bead buoyancy using the Archimedes' principle, from the density of the solution (measured with a Mettler Toledo portable density meter, Densito 30PX), and from the density and the volume of the beads and (2) estimate it from the measured velocities with which the beads settle down. The former and the latter approach provided values for gravitational force of 0.1 pN and 0.3 pN, respectively, that each suspended bead experienced.

The dependence of the magnetic force on the horizontal distance from the magnet core and on the vertical distance from the center of the magnet was tested using a 2-factor ANOVA as implemented by Igor Pro (version 6.22A).58 The side field of view of the bead suspension was separated into four vertical and three horizontal sections, and the velocities of beads in any of the 12 quadrants were measured. From the measured velocities, we calculated the magnetic forces exerted on the beads (Table 3). The two null hypotheses for the ANOVA test were that the magnetic pulling force did not depend on the horizontal position, x, and on the vertical position, y. The p values obtained from the 2-factor ANOVA were px=0.44, py=0.030, and pxy=0.95, not allowing the rejection of the lack of dependence on x, but allowing the rejection of the lack of dependence on y (assuming α=0.05). This finding indicated that all beads within the horizontal field of view (in work mode) experienced the same force.

Measuring Desorption of Beads from Glass Surfaces. For a typical adhesion experiment, we placed the calibrated electromagnet ˜1.5 mm from the surface of the glass (Scheme 1a) and applied 12 V to achieve 1.5 pN of magnetic force. Accounting for the opposing gravitational force provided an estimate of 1.2±0.3 pN for net force pulling upward, which each bead experienced when the magnet was turned on.

Using the magnetic puller in a work mode, we injected a suspension of beads into a PDMS well on the glass slide. By focusing the objective at the surface of the glass, we observed only the beads that settled on the surface, thereby disregarding the beads that had not made contact with the glass.

After allowing the beads to settle on the glass bottom of the well for 3 min, we turned on the electromagnet to exert a relatively weak net force (˜1.2 pN) pulling upward, away from the glass surface. Using a CCD camera (at 10 frames per second), we recorded the beads on the surface from 3 to 5 s before the electromagnet was switched on, to 8-10 s after. The movies were saved as stacks of images and the number of beads remaining on the surface with respect to time was analyzed using Imaris Bitplane software.

The data demonstrate the potential application of the kits and methods for detection, identification, and quantification of multiple bacterial, viral and parasitic targets. The assay revealed a high diagnostic sensitivity and specificity. The summation of these results suggest that this assay could be employed for analysis of patients' blood samples, donor screening for blood-borne pathogens, and investigating the epidemiological trend of infectious pathogens in endemic regions.

It should be recognized that the above described embodiments to which the principles of the disclosure applies are possible examples of illustrative implementation, set forth to provide a clear understanding of this disclosure, and should not be considered as limiting the scope of the invention. Many other variations and further modifications are intended to be made to the above described embodiments without departing from the scope of this invention, rather intended to be included within the scope of this disclosure as well as defined and protected by the following claims.

What is claimed is:

1. A method for real-time multiplex detection and identification of one or more pathogens using glass well plates that include chemically modified surfaces to capture the presence of one or more pathogens from a small sample.

2. A method comprising a glass plate with a chemically modified surfaces and magnetic microbeads with modified surfaces to detect and identify the presence of a bacterial pathogen.
